(19) United States
(12) Patent Application Publication
(10) Pub. No.: US 2009/0270879 A1
(43) Pub. Date: Oct. 29, 2009

(54) Newborn baby shoulder cup extractor

(76) Inventor: Kun Yuan Tong, Suwanee, GA (US)

Correspondence Address: Kun Yang Tong, 2842 Lowe Court, Suwanee, GA, 30024, US

(21) Appl. No.: 12/150,065

(22) Filed: Apr. 24, 2008

(51) Int. Cl. A61B 17/42

(52) U.S. Cl. 606/123

(57) ABSTRACT

During deliver of a baby unexpected shoulder dystocia will be encountered. Shoulder dystocia will cause temporarily or permanent paralysis of upper Extremity or Erb's syndrome. Nowadays vacuum cup extractor is available for obstetricians to deal with mild head dystocia and nothing is available for obstetricians to deal with shoulder dystocia. Newborn baby shoulder cup extractor can assist obstetrician to deliver baby's shoulder when shoulder dystocia is encountered. Newborn baby shoulder cup extractor can prevent the injury to brachial plexus to cause paralysis of upper extremity and unnecessary Cesarean section. After failure of two attempts to deliver baby's shoulder then obstetrician can perform Cesarean section to deliver baby abdominally.

### THE BACKGROUND OF THE INVENTION

In obstetrics there is vacuum cup extractor to help obstetrician to deliver baby's head for difficulty delivery. This vacuum extractor is designed to apply to baby's head for mild head dystocia. There is nothing available for obstetrician to deal with shoulder dystocia. When shoulder dystocia is encountered obstetrician always tries to pull baby's head to deliver baby's shoulder. If too much force is applied to pull baby's head brachial plexus will be injured to cause temporarily or permanent paralysis of upper extremity-Erb's syndrome. It is a disaster to baby and obstetrician. The present invented baby's shoulder cup extractor can be applied to baby's shoulder to allow obstetricians to pull baby's shoulder to prevent the jury to brachial plexus to cause paralysis of upper extremity.

### THE BRIEF DESCRIPTION OF THE INVENTION

When obstetricians encounter shoulder dystocia they can apply newborn baby shoulder cup extractor onto baby's shoulder to deliver baby's shoulder to prevent the injury to brachial plexus to cause Erb's syndrome.

### THE DETAILED DESCRIPTION OF THE DRAWINGS

FIG. 1 is a perspective view of newborn baby shoulder cup extractor.

FIG. 2 is a perspective view of vacuum cup extractor.

FIG. 3 is a perspective view of the bottom of newborn baby shoulder cup extractor.

FIG. 4 is lateral view of newborn baby shoulder cup extractor.

FIG. 5 is the dissection view of the newborn baby shoulder cup at midline. extractor.

### THE DETAILED DESCRY[TION OF THE INVENTION

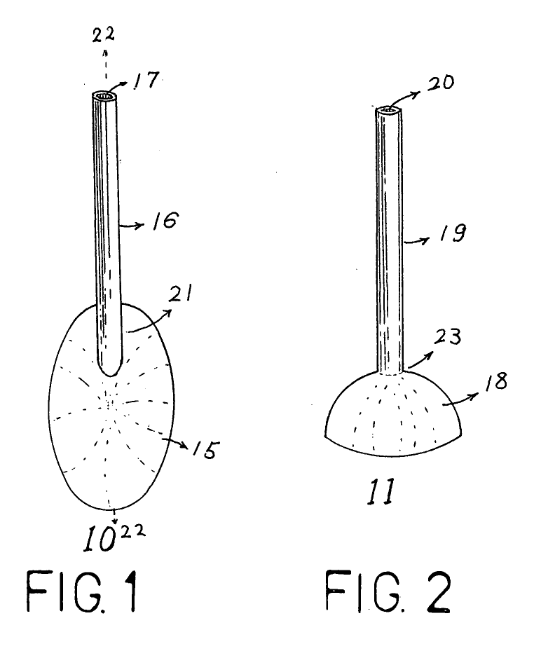

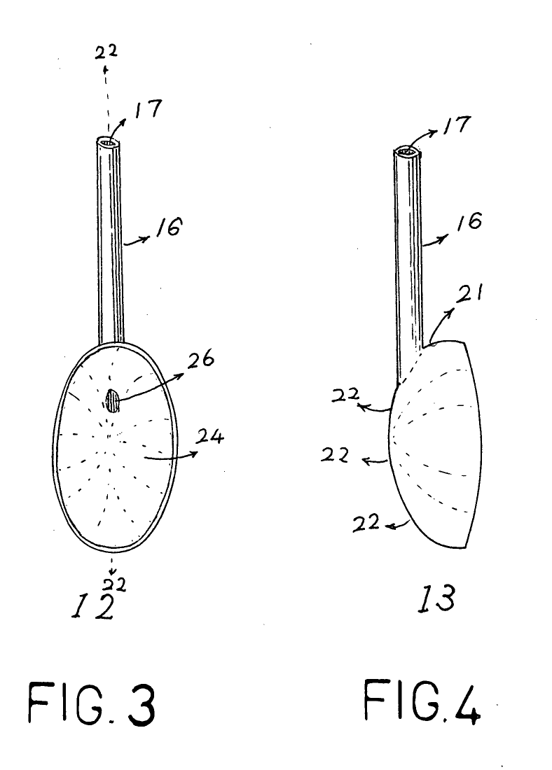

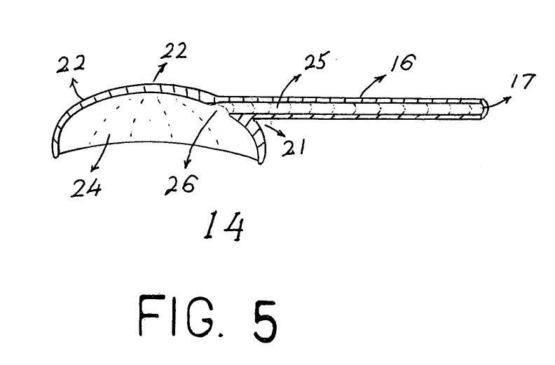

Referring now in detail to the drawings numeral 10 of FIG. 1 is newborn baby shoulder cup extractor. Newborn baby shoulder cup extractor 10 is applied to baby's shoulder to help obstetrician deliver baby's shoulder when shoulder dystocia is encountered. Numeral 11 of FIG. 2 is vacuum cup extractor. Obstetrician applies vacuum cup extractor 11 onto baby's head to deliver baby's head when difficulty delivery is encountered. Numeral 12 of FIG. 3 is the bottom view of newborn baby shoulder cup extractor 10. Numeral 13 of FIG.4 is the lateral view of newborn baby shoulder cup extractor 10. Numeral 22 of FIGS. 1, 3,4 and 5 is the midline of newborn baby shoulder cup extractor 10. Numeral 14 of FIG. 5 is the dissection view at the midline 22 of newborn baby shoulder cup extractor 10. Numeral 15 of FIG. 1 is shoulder cup. Shoulder cup 15 is of oval-shaped and is of a variety of size. Shoulder cup 15 fits baby's shoulder very well. Shoulder cup 15 does not fit baby's head. Numeral 16 of FIGS. 1, 3, 4 and 5 is hollow handlebar of newborn baby shoulder cup extractor 10. Numeral 17 of FIGS. 1, 3, 4 and 5 is external opening of hollow handlebar 16. Numeral 18 of FIG. 2 is head cup of vacuum cup extractor 11. Head cup 18 is of round-shaped and fits baby's head very well. Head cup 18 does not fit baby's shoulder well. Numeral 19 of FIG. 2 is hollow handlebar of vacuum cup extractor 11. Numeral 20 of FIG. 2 is external opening of vacuum cup extractor 11. Numeral 21 of FIGS. 1, 4 and 5 is the side wall along midline 22 of shoulder cup 15. Hollow handlebar 16 is attached to the side wall 21 of newborn baby shoulder cup extractor 10. Numeral 23 is the dome of vacuum cup extractor 11. Hollow handlebar 19 is attached to the dome 23 of head cup 18. The difference between newborn baby shoulder cup extractor 10 and vacuum cup extractor 11 are the shape and location of attachment of hollow handlebar. Shoulder cup 15 is of oval-shaped and head cup 18 is of round-shaped. Hollow handlebar 16 is attached to side wall 21 along midline 22 of newborn baby shoulder cup extractor 10 and hollow handlebar 19 is attached to dome 23 of head cup 18 of vacuum cup extractor 11. Numeral 24 of FIGS. 3 and 5 is chamber of shoulder cup 15. Numeral 25 of FIG. 5 is canal of hollow handlebar 16. Numeral 26 of FIGS. 3 and 5 is internal opening of canal 25. When shoulder dystocia is encountered during the delivery obstetrician can perform episiotomy if it has not been done and applies shoulder cup 15 of newborn baby shoulder cup extractor 10 onto baby's shoulder to deliver baby's shoulder. After failure of two attempts to deliver baby's shoulder obstetrician can perform Cesarean section to deliver baby abdominally to prevent the injury to brachial plexus to cause Erb's syndrome.

1. Newborn baby shoulder cup extractor comprising an oval-shaped cup and an attached hollow handlebar.

2. The said oval-shaped cup in claim 1 has an oval-shaped opening that is of a variety of size to fit different size of baby's shoulder and is made of soft flexible material. In shape, the said oval-shaped cup is like half of the shell of an egg that is cut into two exact halves along the longitudinal line.

3. The said attached hollow handlebar in claim 1 is attached to the side of oval-shaped cup in claims 1 and 2 and has a canal that its internal opening connects to the chamber of the said oval-shaped cup and its external opening can be connected to a suction machine with a tube.

4. In shape, the said newborn baby shoulder cup extractor in claim 1 is like an ice cream scoop.
